(19) United States
(12) Patent Application Publication
(10) Pub. No.: US 2022/0186168 A1
(43) Pub. Date: Jun. 16, 2022

(54) ROTATING SUSPENSION CULTURE DEVICES THAT ALLOW DIRECT MICROSCOPY, IN SITU ASSAYS, AND AUTOMATION

(71) Applicant: Cell Spinpod LLC, Chapel Hill, NC (US)

(72) Inventors: Holly H. Birdsall, Chapel Hill, NC (US); Timothy G. Hammond, Chapel Hill, NC (US)

(21) Appl. No.: 17/118,638

(22) Filed: Dec. 11, 2020

(51) Int. Cl. C12M 3/06; C12M 1/26; C12M 1/00

(52) U.S. Cl. CPC C12M 27/14; C12M 23/22; C12M 33/18

(57) ABSTRACT

Rotating suspension culture devices that allow direct microscopy, in situ assays, and automation are disclosed. According to an aspect, a suspension culture device includes a rotatable base having an exterior surface that engages at one or more rollers for rotation of the base about an axis when the at least one roller is turning. The device includes first and second end components attached to the base along the axis. The base and the first and second end components define an interior space for holding liquid. A portion of at least one of the end components is made of a material that is at least partially transparent for viewing into the interior space from outside the base. Further, the device includes ports that each permit fluid communication between the interior space and outside the base.

### TECHNICAL FIELD

The presently disclosed subject matter relates generally to devices for biologic studies. Particularly, the presently disclosed subject matter relates to rotating suspension culture devices that allow direct microscopy, in situ assays, and automation.

### CROSS REFERENCE TO RELATED APPLICATIONS

The present application is related to U.S. Design Patent Application titled SUSPENSION CULTURE DEVICE and another U.S. Design Patent Application titled SUSPENSION CULTURE DEVICE, each filed simultaneously herewith.

### BACKGROUND

Cells in the body are exposed to flow shear as fluids, such as blood or renal glomerular filtrate, flow past the outer cell membrane. In the kidney, fluid from the blood is filtered in the glomerulus and this ultrafiltrate flows past proximal tubule cells (PTC) that are responsible for reabsorbing water, sodium, glucose, amino acids, and diverse hormones and proteins. This is not a languid process—the kidney generates over 100 ml of ultrafiltrate per minute and the proximal tubule cells are responsible for reabsorbing 70% of this volume. Calculations of the fluid shear stress in vivo is complicated by the varying dimensions of the tubules and varying composition of the ultrafiltrate as it moves down the tubule, but it is estimated that PTC are exposed to shear stress in the range of 0.04-2 dynes/cm². Higher level of fluid shear stress can occur during renal dysfunction and are thought to contribute to disease progression and play a key role in progression of chronic renal disease.

Fluid shear stresses have an important role in maintaining the differentiation of PTC. To be meaningful and representative models of living kidneys, cultured proximal tubule cells must be exposed to fluid shear stress in vitro. Exposure to fluid shear stress increases PTC transport of proteins, expression of microvilli, and formation of tight junctions with increased transepithelial electrical resistance.

Several limitations may account for discrepancies between the many published reports on the effects of flow shear stress on cultured PTC. Some investigators use primary PTCs from human, rat, or mouse kidneys. Others use cell lines such as H2K, MDCK, RPTEC/TERT1, OK opossum kidney cell line, or SV40 transformed proximal tubular epithelial cells (PTEC), each of which varies as to how well they maintain the function of in vivo cells. The shear stresses employed in these studied range from 0.02 to 9.0 dynes/cm² and are applied for times ranging from 15 minutes to over two weeks. Finally, the flow shear stress is applied using diverse technologies including orbital shakers, parallel plates, microfluidics with peristaltic pumps, and perfused hollow fibers. Many of these options require expensive equipment and are rarely practical for evaluating large numbers of replicates due to cost and vessel volume.

Suspension cultures, in which cells float in a liquid milieu, have significant advantages for the delivery of physiologic levels of flow shear stress. Suspension culture technology has been modelled, validated experimentally, and matured for routine use. Roller bottles, paddle stirrers, and shakers are inexpensive options and quite suitable for fungi, bacteria, and algae that can tolerate high shear levels and are relatively resistant to injury from impact against the vessel walls. But mammalian cells need much gentler treatment to avoid cellular damage and to mimic the shear levels they experienced in vivo.

Rotating suspension cultures can provide physiologic levels of fluid shear stress. Controlled shear is achieved by zero head space, that is filling the vessel entirely with culture media, so that the contents rotate in laminar flow and avoid turbulent flow entirely. The rotating wall vessel spins around a horizontal axis and the cells move in an annulus around the axis of rotation. Cells and aggregates of different size and density co-localize in the annulus. Cells do not need to adhere to a plastic surface and thereby avoid the de-differentiation associated with 2D cultures. However, cells can be attached to beads or other scaffolds, as needed. A gas permeable membrane allows for gas exchange.

Rotating suspension culture has found limited applicability due to limitations of the currently available hardware. Re-usable vessels have multiple components needing autoclaving at different temperatures, as well as manual assembly in a cell culture hood. The vessels attach to spindle rotators that spin with great precision. However, the rotators are expensive and can only hold a few vessels. Commercial applications are largely limited to generation of large numbers of tissue spheroids that are transferred to other systems for experimentation.

In view of the foregoing, there is a dire need for small suspension culture devices that are affordable, simple to use, and adaptable for use in studies with large numbers of replicates. Further, there is a parallel need for larger affordable, simple to use suspension culture devices to produce commercial quantities of biomolecules.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is an oblique perspective view of an example suspension culture 3.2 ml device in accordance with embodiments of the present disclosure;

FIG. 2 illustrates a side view of the device shown in FIG. 1;

FIG. 3 is an oblique perspective view of an example suspension culture 3.2 ml device showing the opposite side to FIG.1;

FIG. 4 Illustrates a side view of the device shown in FIG. 1 1, from the opposite side to FIG. 2;

FIG. 5 is an oblique exploded view of the base including the silicone rubber material for the ports of the suspension culture device shown in FIG. 1;

FIG. 6 is an oblique exploded view of the base including the silicone rubber material for the ports of the suspension culture device shown in FIG. 1 from the opposite side to FIG. 5;

FIG. 7 is FIG. 6 is an en face exploded view of the suspension culture device shown in FIG. 1;

FIG. 8 illustrates a middle section of the device shown in FIG. 1;

FIG. 9 is a side view of the device shown in FIG. 1, sitting in the loading dock, with an air bleed needle, and loading butterfly in place, as a person's hand holds the syringe delivering reagents;

FIG. 10 is an oblique view of the device shown in FIG. 1 sitting in the loading dock;

FIG. 11 shown the device shown in FIG. 1 sitting in the microscopy dock;

FIG. 12 shows the component of the microscopy holder for the device shown in FIG. 1;

FIG. 13 is an oblique view of another example suspension culture device in accordance with embodiments of the present disclosure;

FIG. 14 is a side view of the device shown in FIG. 13;

FIG. 15 is an oblique view of the device shown in FIG. 13 from the opposite side to FIG. 13;

FIG. 16 is an oblique exploded view of the base including the silicone material for the ports of the suspension culture device shown in FIG. 13;

FIG. 17 is an en face exploded view of the suspension culture device shown in FIG. 13; and

FIGS. 18A and 18B show respectively the volume fraction of particles and velocity vectors illustrating the suspension flow, and the distribution of the magnitude of the deviatoric stress tensor of the particle phase.

### SUMMARY

The presently disclosed subject matter relates to rotating suspension culture devices that allow direct microscopy, in situ assays, and automation. According to an aspect, a suspension culture device includes a rotatable base having an exterior surface that engages at one or more rollers for rotation of the base about an axis when the at least one roller is turning. The device includes first and second end components attached to the base along the axis. The base and the first and second end components define an interior space for holding liquid. A portion of at least one of the end components is made of a material that is at least partially transparent for viewing into the interior space from outside the base. Further, the device includes ports that each permit fluid communication between the interior space and outside the base.

According to another aspect, a suspension culture system includes one or more rollers. Further, the system includes a mechanism configured to turn the rollers. The system also includes a suspension culture device including a rotatable base having an exterior surface that engages the roller(s) for rotation of the base about an axis when the at least one roller is turning. Further, the system includes first and second end components attached to the base along the axis, wherein the base and the first and second end components define an interior space for holding liquid, wherein a portion of at least one of the end components is made of a material that is at least partially transparent for viewing into the interior space from outside the base. Further, the system includes ports that each permit fluid communication between the interior space and outside the base.

According to another aspect, an adaptor is disclosed for holding a suspension culture device for observation of contents of the suspension culture device. The adaptor includes a base portion comprising a top portion defining a surface and a bottom portion defining a surface. Further, the base portion defines an aperture that extends between the surface of the top portion and the surface of the bottom portion. The adaptor includes a suspension culture device holder comprising a first feature and a second feature. The first feature is configured for holding a suspension culture device. The second feature is configured for fitting to the aperture of the base portion.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

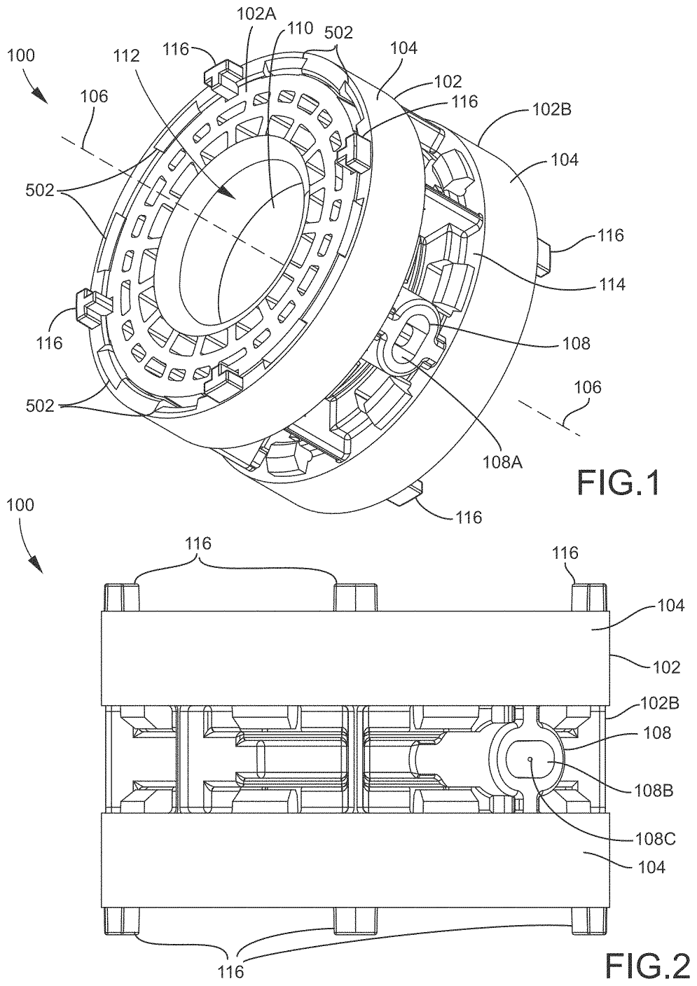

FIG. 1 illustrates a perspective view of an example suspension culture device 100 in accordance with embodiments of the present disclosure. In this example, the device 100 includes a rotatable base 102 having one or more exterior surfaces 104. The rotatable base 102 may be substantially shaped as a disk or any other suitable shape such that it can suitably engage with one or more rollers for rotation of the rotatable base 102. The device 100 has a capacity in its interior space of approximately 3.5 ml. In this example, the rotatable base 102 includes an axis of rotation, which is represented by broken line 106, around which the rotatable base 102 can rotate when moved by the rollers as described in further detail herein. The rotatable base 102 may define an interior space (not shown in FIG. 1) for holding a culture. When the rotatable base 102 is rotated, the culture can be kept in suspension such that it does not settle at the bottom of the interior space. The culture may be a cell culture medium. Also, the interior space may hold, for example, one or more of support structures, beads, test substances, drugs, peptides, and viruses.

In accordance with embodiments of the present disclosure, some or the entirety of the rotatable base 102 may be made of a breathable material that extends between the interior space and outside the rotatable base 102. As a result, oxygen or other gas from outside the rotatable base 102 may enter into the interior space to thereby allow cells in the culture to maintain their metabolism and differentiation. Further, gases such as carbon dioxide, produced by cell metabolis, can escape the interior space. The breathable material is selected for differential gas exchange such that water is retained orders of magnitude better, than oxygen and carbon dioxide are diffused. In some embodiments, the rotatable base 102 has one or more portions that are thinner than other portions to provide an easier pathway for oxygen from the outside into the interior space. These portions of the rotatable base 102 can be any suitable size, shape, and provide any suitable thickness between the outside and the interior space. An example of these portions is 0.001″ thick FEP (fluorinated ethylene propylene). In examples, these portions can take the form of divots, indentations, and the like in the base 102. Moreover, the rotatable base may be designed for controlling an amount of oxygenation, carbon dioxide removal, and water retention, desired within the interior space where the culture is located.

The breathable material of the base 102 may be any suitable material that permits gas to pass through it. Example breathable material includes, but is not limited to, fluoroplastic, fluorinated ethylene propylene (FEP), PerFluoroAlkoxy (PFA), polytetrafluoroetylene (PTFE), the like, and combinations thereof.

The device 100 shown in FIG. 1 includes multiple ports 108 (only one of which is shown in FIG. 1) that each permit fluid communication between the interior space and outside the rotatable base 102. In particular, a port 108 may be used for introducing culture into the interior space of the rotatable base 102. A port 108 may also be used for removing air, another gas, or liquid from the interior space of the rotatable base 102.

In this example, a port 108 is made of a silicone rubber material that is positioned within a hole defined in the base 102. The hole provides a passageway that extends from outside the base 102 to the interior space. A blunt or sharp (sharp needle hole can seal better) needle (e.g., 18 to 26 gauge blunt or sharp needle) or other suitable instrument may penetrate the rubber material of the port 108 such that liquids can be introduced into the interior space. Once the needle is removed, the rubber material may reseal the port 108. Air may be bled from the interior space by use of another needle at another port.

As shown in FIG. 1, the ports 108 are positioned at or near an outer edge of the base 102. Particularly, in this example the ports 108 are positioned between 2 exterior surfaces 104. Alternatively, the ports 108 may be positioned at any other suitable area of the base 102.

The device 102 also includes multiple windows 110 attached to the base 102 for permitting viewing into the interior space. For example, cells in the interior space may be stained with fluorescent dyes and imaged by inverted fluorescent microscopy. In this example, the base 102 defines multiple apertures 112 that lead to where respective windows 110 are positioned. The contents in the interior space may be observed by viewing through an aperture 112 and its respective window 110. The windows 110 are sealed such that fluid cannot escape from the interior space. Further, the windows 110 may be made of transparent, semi-transparent, or substantially transparent such that a person or instrument may see through the window 110 into the interior space. In an example, the windows 110 may be made of FEP and have a thickness of between about 0.0005″ and 0.05″. As in described herein, example breathable material includes, but is not limited to, fluoroplastic, fluorinated ethylene propylene (FEP), PerFluoroAlkoxy (PFA), polytetrafluoroetylene (PTFE), the like, and combinations thereof.

Alternatively, the material of the base 102 may be partially or entirely transparent such that the contents of the interior space can be viewed from the outside.

It is noted that the components may be made by 3D printing or any other suitable technique, such as injection molding. Examples include, but are not limited to, FEP and PFA techniques.

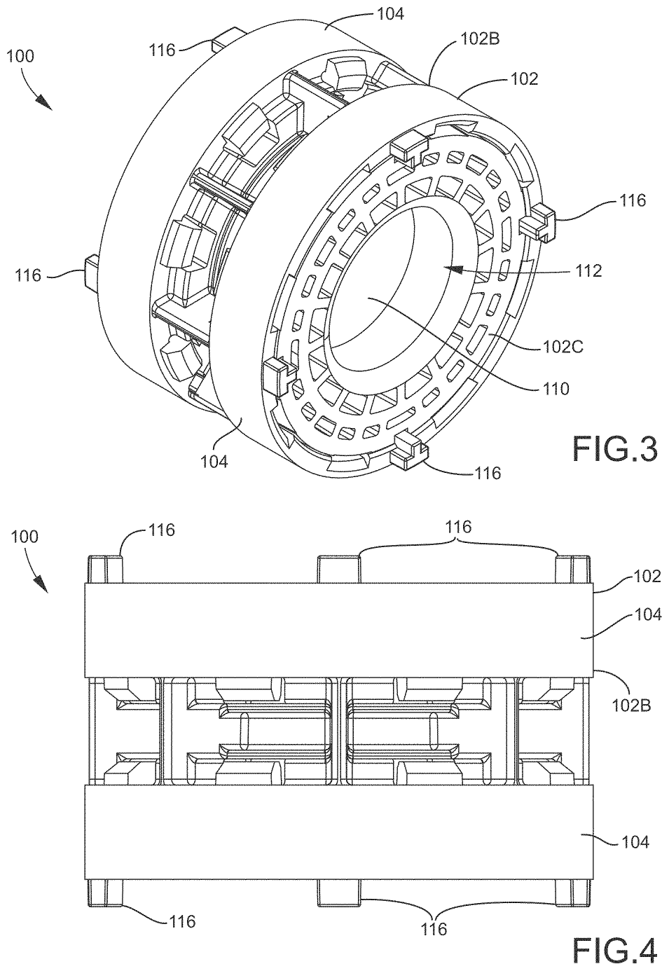

FIGS. 2, 3, and 4 illustrate a side view, another perspective view, and another side view of the suspension culture device 100 shown in FIG. 1. The perspective view shown in FIG. 3 is from a different end of the device 100.

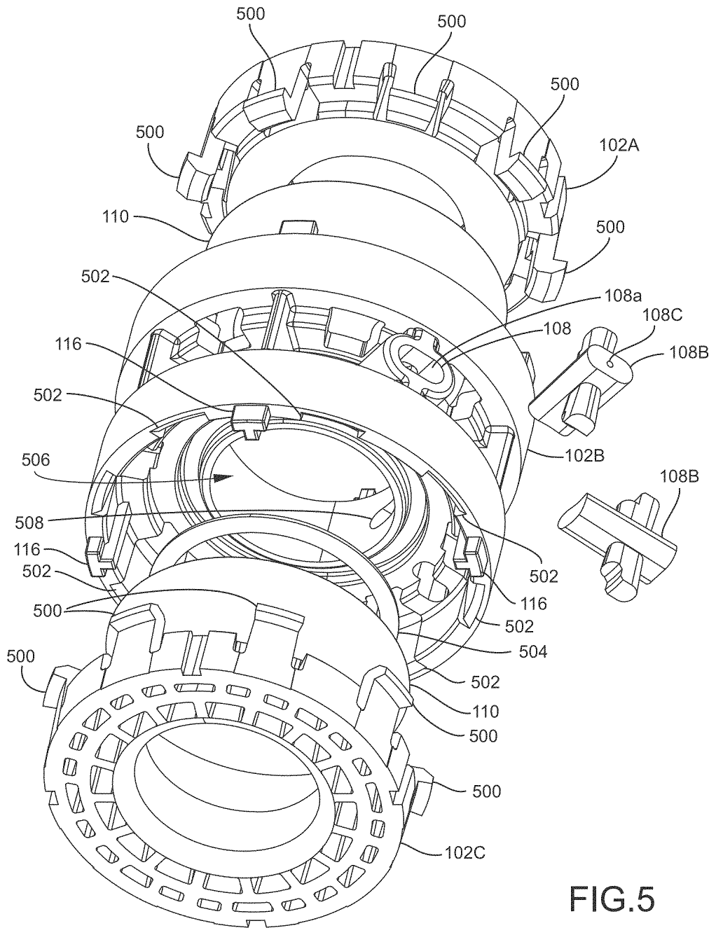

FIG. 5 illustrates an exploded view of the suspension culture device 100 shown in FIGS. 1-4. Now referring to FIG. 5, the base 102 of the device 100 includes three (3) components that can be assembled together as shown in FIGS. 1-4. Particularly, these components include a top component 102A, a middle component 102B, and a bottom component 102C. In this example, the top component 102A can be securely attached to the middle component 102B via multiple, cantilever snap-fits 500. The top component 102A can be moved in the direction of the middle component 102B and oriented such that the cantilever snap-fits 500 can engage mating parts of the middle component 102B and attach the top component 102A to the middle component 102B.

The bottom component 102C also has cantilever snap-fits 500 and can be moved towards the middle component 102B to similarly attach to the middle component 102B on its opposing side. As shown in FIG. 5, the snap-fits 500 of the bottom component 102C can engage guide features 502 of the middle component 102B and continue movement into the middle component 102B until the tips of the snap-fits 500 engage and lock to a corresponding internal feature of the middle component 102B. Now turning again to FIG. 1, the tips of the snap-fits 500 are shown as being locked to an internal ridge 114 of the middle component 102B for attaching the bottom component 102C to the middle component 102B.

As shown in FIGS. 1-4, the top component 102A and the bottom component 102C fit inside opposing ends of the middle component 102B. The device 100 also includes a gasket 504 that provides a seal between the corresponding lens 110 and the middle component 102B when the device 100 is assembled as shown in FIGS. 1-4. There is another gasket (not shown) at the opposing end that provides a seal between the other lens 110 and the middle component 102B when the device 100 is assembled as shown in FIGS. 1-4. This assembly forms the interior space 506 for holding the culture. Also, in FIG. 5, an opening 508 of one of the ports 108 is shown. The opening leads through the port 108 to outside the device. The gaskets 504 seal the windows 110 to the middle component 102B such that the culture does not leak from the interior space 506.

It is noted that although cantilever snap-fits 500 are used in this example as attaching the components 102A, 102B, and 102C together, it should be understood that any other suitable mechanism may be used for attaching the components 102A, 102B, and 102C together.

Ports 108 each include an aperture 108A and a pliable material 108B that fits into the aperture 108B. The pliable material 108B can be made of silicone rubber and defines a passageway 108C that extends between outside the base 102 to the interior space 506. The passageway 108C may be used for introducing culture into the interior space 506 or removing air, another gas, or liquid from the interior space 506.

With continuing reference to FIGS. 1-5, the device 100 include multiple protrusions 116 that extend from ends of the device 100. The functionality of the protrusions is that they both provide space for the devices to breath, and engage so that a group of devices on a roller will turn in exact unison,

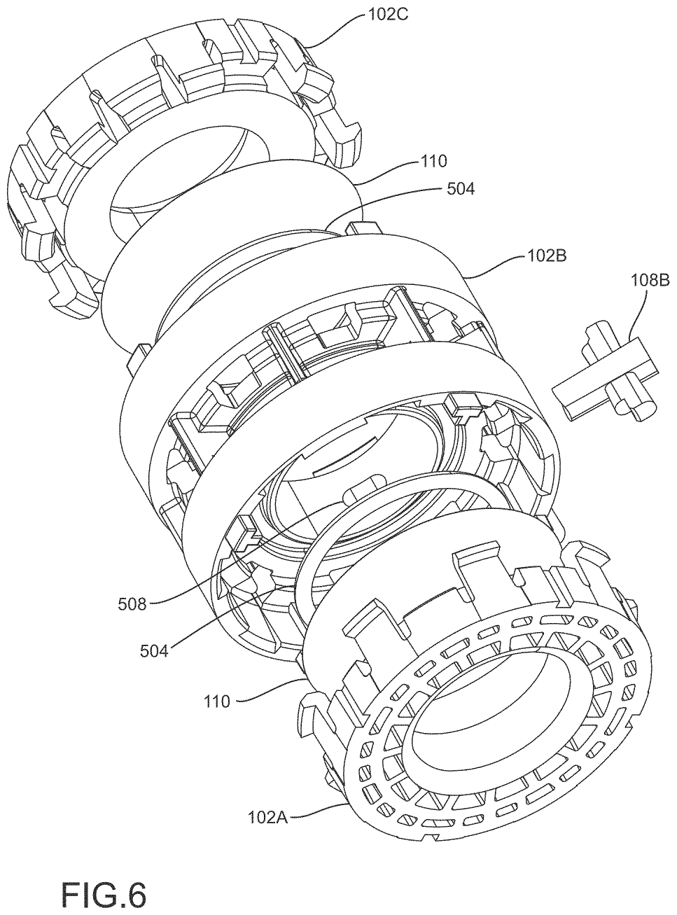

FIG. 6 illustrates another exploded view of the suspension culture device 100 of FIGS. 1-5 from another end of the device as shown in FIG. 5.

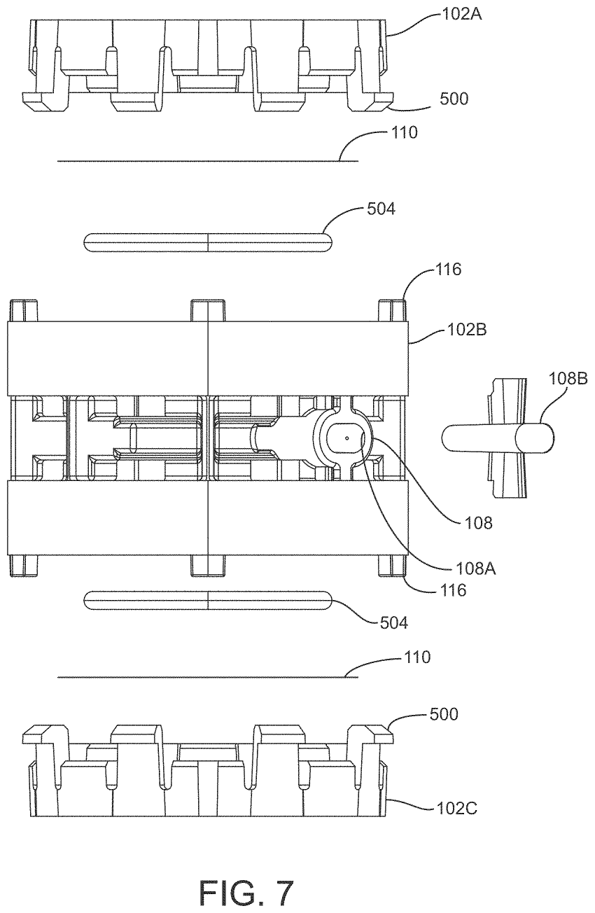

FIG. 7 illustrates an exploded side view of the suspension culture device 100 of FIGS. 1-6.

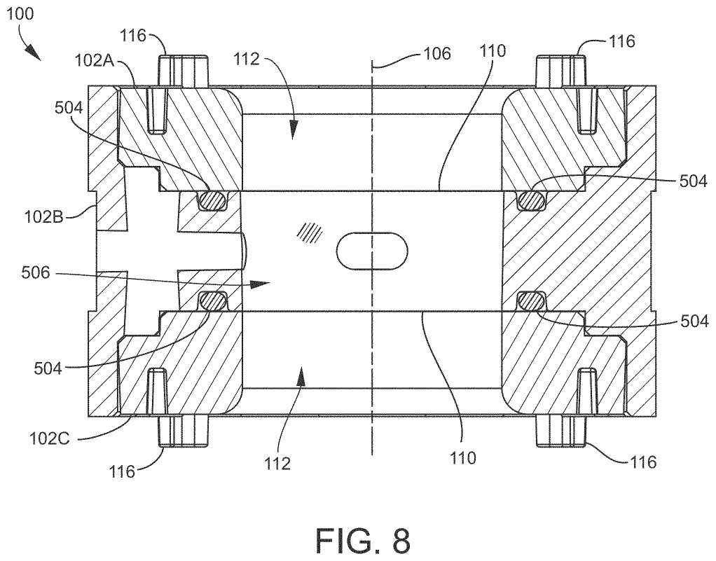

FIG. 8 illustrates a cross-sectional side view of the suspension culture device 100 shown in FIGS. 1-7.

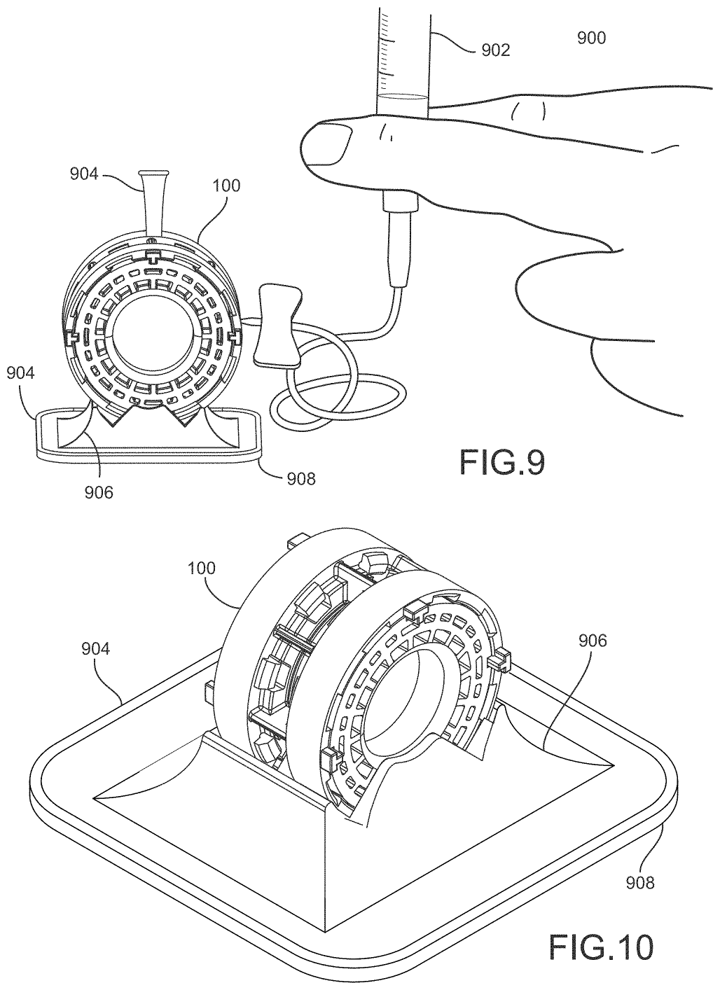

FIG. 9 illustrates a side view of a person's hand 900 holding a hypodermic needle 902 and injecting fluid into a port of the suspension culture device 100 shown in FIGS. 1-8. Referring to FIG. 9, the fluid is injected into the interior space of the device 100. Also, an apparatus 904 is interfaced at another port of the device 100 for removing or “bleeding” air from the interior space. As shown, the port receiving the culture is positioned at the side, and the port where the air exits is at the top to more easily receive the culture and remove the air. The air may be removed by a suitable needle, such as a 26 gauge needle.

With continuing reference to FIG. 9, the device 100 is being held upright by the stand 904. As shown, the lower part of the device 900 rests on a top portion 906 of the stand 904 that is shaped and sized to conform to the device 900. A bottom portion 908 of the stand has a wide dimension to provide stability to the top portion 906 and the device 900.

FIG. 10 illustrates another perspective view of the suspension culture device of FIGS. 1-8 being held by the stand 904.

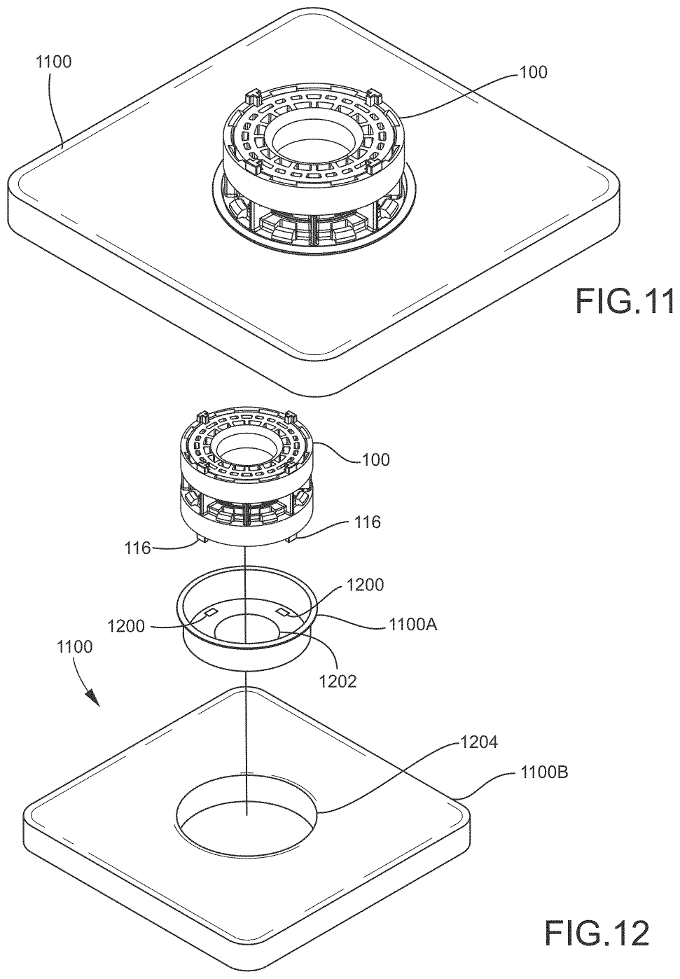

FIG. 11 illustrates a perspective view of the suspension culture device 100 being held by an adaptor 1100 and positioned for observation of its culture by a microscope in accordance with embodiments of the present disclosure. As shown, the device 100 is held on its side. Although not shown in FIG. 11, the adaptor 1100 defines an aperture (not shown) so that the lower window of the device 100 is viewable through the aperture. The edge of the aperture is shaped and sized to let the outside edge of the lower part of the device 100 to be held thereby.

FIG. 12 illustrates an exploded view of the assembly of the device 100 and the adaptor 1100. Referring to FIG. 12, the adaptor 1100 has two components 1100A and 1100B. The component 1100A can hold the device 100 and has apertures 1200 to receive the protrusions 116 of the device 100. The component 1100A can fit into the component 1100B. Also, the components 1100A and 1100 each define apertures 1202 and 1204, respectively, that align with each other for forming the aforementioned aperture for the aforementioned viewing of the lower window of the device 100.

The use of the adaptor 1100 with the device 100 brings the device into the focal length of lenses commonly in use on inverted microscopes.

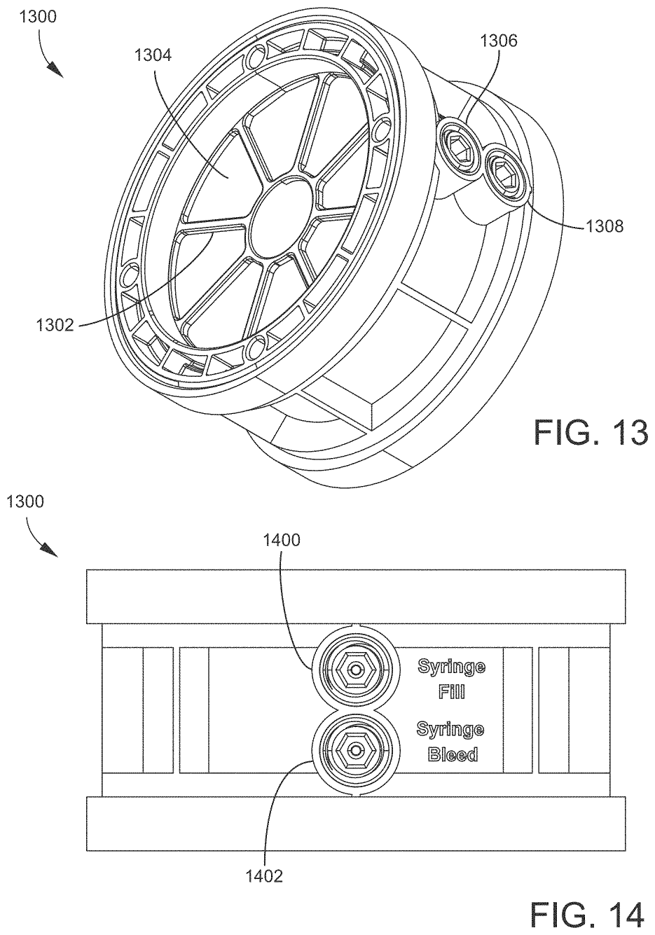

FIG. 13 illustrates a perspective view of another example suspension culture device 1300 in accordance with embodiments of the present disclosure. The device 1300 has a capacity in its interior space of approximately 250 ml. The device 1300 in this example is similar to the device 100 of FIGS. 1-12 except that the device 1300 is sized differently, has frames 1302 to support and protect its windows 1304, and has 4 ports rather than 2 ports. In the view of FIG. 13, 2 ports 1306 and 1308 are shown. The other 2 ports are positioned on an opposing side of the device 1300 and therefore not shown in this view.

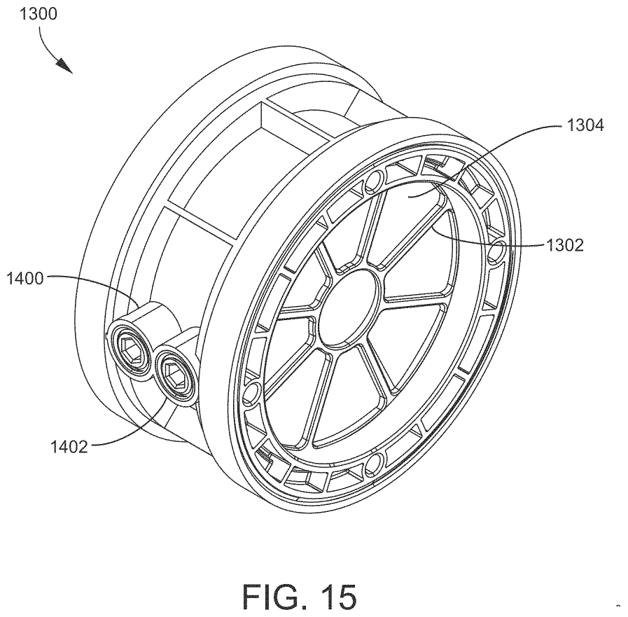

FIG. 14 illustrates a side view of the suspension culture device 1300 shown in FIG. 13. Referring to FIG. 14, ports 1400 and 1402 are shown, and these ports are on an opposite side of the device than ports 1306 and 1308 shown in FIG. 13. FIG. 15 illustrates another perspective view of the suspension culture device that shows ports 1400 and 1402.

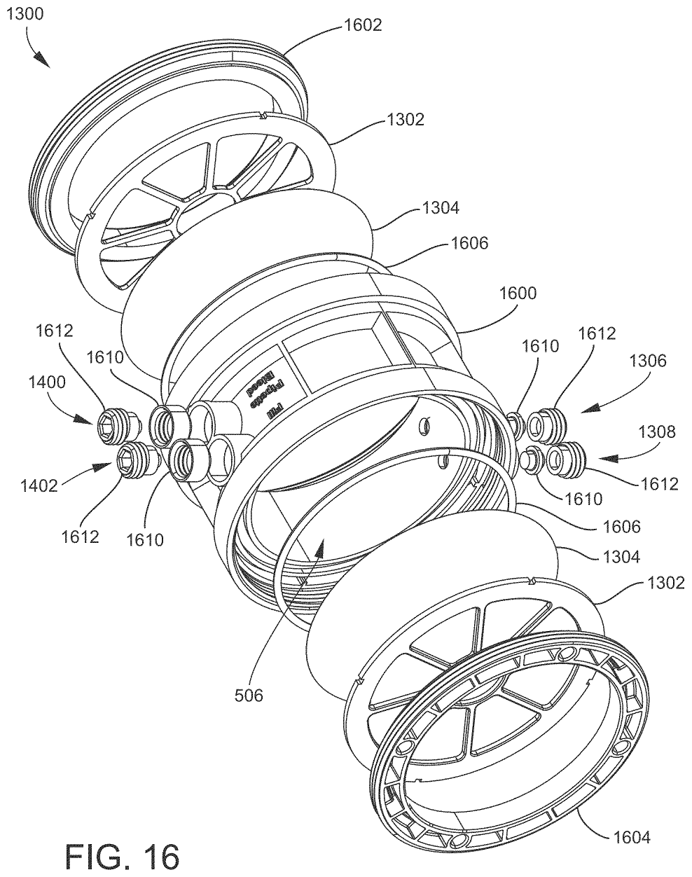

FIG. 16 illustrates an exploded view of the suspension culture device 1300 shown in FIGS. 13-15. Now referring to FIG. 16, the device 1300 includes a body 1600 and opposing end caps 1602 and 1604. The end caps 1602 and 1604 can attach to the body 1600 in the assembled positions shown in FIG. 13. Also, as assembled, each cap 1602 and 1604 can securely hold in place its respective frame 1302, window 1304, and gasket 1606. When assembled, each gasket 1606 provides a seal between its corresponding window 1304 and the body 1600. A cap 1602 can be “snap” fitted to attach to the body 1600.

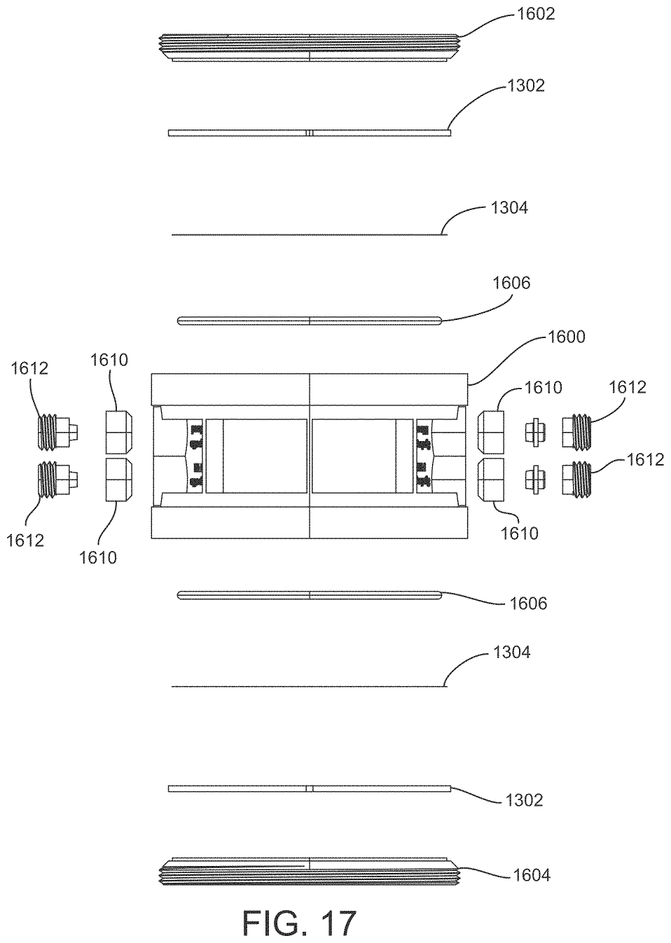

With continuing reference to FIG. 16, each port 1306, 1308, 1400, and 1402 includes an aperture 1608 that leads to the interior space 506 where a culture can be held for experiments. Further, each port 1306, 1308, 1400, and 1402 includes a pliable material 1610 that can be made of silicone rubber and that defines a passageway that extends between outside the body 1600 to the interior space 506. Further, each port 1306, 1308, 1400, and 1402 includes “snap” component 1612 that can connect to the body 1600 for holding its respective pliable material 1610 in place. The ports 1402 of FIG. 16 are large enough to accommodate large laboratory pipettes for efficient filling of the large device shown in FIG. 13. The 130 and 1308 ports shown in FIG. 16 are small to allow needle removal of any residual air bubbles, facilitating maintenance of laminar flow in the vessel. FIG. 17 illustrates a side, exploded view of the suspension culture device 1300 shown in FIGS. 13-16.

For experimentation, the device 100 of FIGS. 1-11 can be used for analytical work. Initially, a device can be placed in a loading docket, such as the apparatus 904 shown in FIG. 9. Subsequently, a sterile 26-gauge needle can be inserted at the port at 12 o'clock. A plunger can be pulled out of a sterile 5 ml syringe. The syringe can be attached to a 19-gauge butterfly. Further, the butterfly needle can be inserted into the cell spinpod port at 3 o'clock. Cells, media, and support beads or drugs can then be added to the 5 ml syringe. Next, the syringe can be raised so the contents slowly fill the device and media can be seen in the hub of the air bleed needle in the 12 o'clock port position. Once the interior space of the device is full, the butterfly needle at 3 o'clock can be removed. Next, the air bleed needle at 12 o'clock can be removed. Next, the device can be placed on a bottle roller prepositioned in a 5% CO₂ incubator. Once all the cell pods are in place, the bottle roller can be turned on to a rate that the cells are visibly rotating in suspension (approximately 12-20 rpm). The device can be rotated for a desired period.

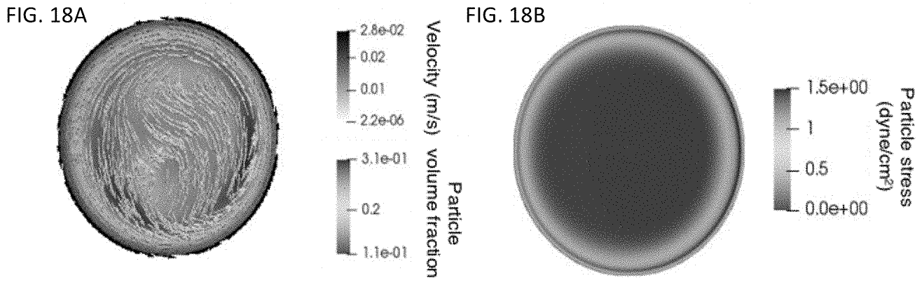

Simulations were performed to evaluate the fluid mechanical forces experienced by cells in the spinpod. FIGS. 18A and 18B show respectively the volume fraction of particles and velocity vectors illustrating the suspension flow, and the distribution of the magnitude of the deviatoric stress tensor of the particle phase. The plots shown are in a cross-section perpendicular to the rotation axis, having first achieved a steady state in the simulation upon starting the rotation from rest. The highest stresses on the particle phase are encountered near the vessel wall (strongest shear) but rapidly decrease to a level of about 0.5 dynes/cm² in the annular region slightly inward from the wall, wherein the volume fraction of particles is highest (about 85%).

The viability of RPTEC/TERT1 renal cells in rotating spinpods was not significantly different from that of static spinpods at the end of three days of culture (FIG. 4). Compared to rotating spinpods, static spinpods tended to have fewer live cells (70±2% vs. 77%±3), more cells in early apoptosis (19±2% vs. 15±2%) and more cells in late apoptosis (12±2% vs. 9±2%), but the differences did not reach statistical significance.

The next generation sequencing shows a different sequence and timing of responses of RPTEC/TERT1 renal cells in spinpods when they are static or rotated (Table I below). At 3 hours the cells in static spinpods are already displaying increases in RNA gene expression and RNA polymerase biosynthesis. There are already cellular changes in cytokine signaling, apoptotic cell death, immune effector defense, and intracellular protein phosphorylation. By 24 hours the cells in static spinpods have large changes in oxygen compound response, and apoptotic process regulation. At the same 24-hour time period, the rotating cells are showing changes in cell cycle regulation, apoptosis, and catabolic processes. Again, this is consistent with our flow cytometry and cytokine data. By 72 hours the cells in static spinpods show changes in DNA metabolic response, oxidation reduction processes, oxidative stress response, cell cycle, and lipid metabolism. At the same 72-hour time point the rotating cells demonstrate changes in response to toxic compounds, cell death regulation, and vessel morphogenesis development.

TABLE I

STATIC SPINPODS
3 hrs. vs 0 hrs.
MA gene expression
MA polymerase biosynthetic
Developmental growth
Intracell. protein phosphorylation
Negative signaling stimulus
Binding factor activity
Response nitrogen compound
Immune effector defense
Negative regulation transport
Response cytokine signaling
Cell population proliferation
Cell death apoptosis
Cell differentiation developmental
Blood morphogenesis devel.
Component movement locomotion
Animal organ morphogenesis
Homeostasis cellular chemical
24 hrs. vs 0 hrs.
Response oxygen compound
Apoptotic process regulation
72 hrs. vs 0 hrs.
Oxidation reduction process
Response oxidative stress
Lipid metabolic process
Response DNA metabolic
Negative cell cycle
ROTATING SPINPODS
3 hrs. vs 0 hrs.
Cell motility regulation
Formation involved morphogenesis
Intracellular signal regulation
Immune system activation
Inflammatory response defense
Response cytokine signaling
Cellular response nitrogen
Cell death apoptotic
24 hrs. vs 0 hrs.
Catabolic macromolecule process
Homeostasis cellular chemical
Small molecule metabolic
Molecular function negative
Cellular response compound
Molecular function negative
Regulation cell cycle
Positive polymerase
Neuron death apoptotic
Regulation cellular stress
72 hrs. vs 0 hrs.
Death regulation cell
Response wounding
Vessel morphogenesis devel.
Extracellular stimulus external
Response toxic compound
Reduction process metabolic
Negative regulation signaling

Notably, of all the common, well-characterized renal transporters, the only one that changed in the rotating spinpods was the breast cancer resistance protein (HGNC Gene Symbol ABC-G2, common symbol BCRP). BCRP was reduced at 3 hours in the static cultures (differential expression q-value 0.031), but this reduction was delayed in rotating cultures with differential expression q-values of 0.036 at 24 hours, and 0.02 at 72 hours respectively. There was no change at any time point in other drug transporters known to be expressed by PCT including Organic Anion Transporter 1 (OAT1), Organic Anion Transporter 3, (OAT-3) Organic Anion Transporter 4 (OAT-4), Urate Anion Exchanger 1, Organic Cation Transporter 2 (OCT-2), Multidrug and Toxin Extrusion Protein 1 (MDR-1, also known as MDR-1 and P-gp), Multidrug Resistance Associated Protein 2 (MDR-2), Multidrug Resistance Associated Protein 2 (MDRAP-1), or Multidrug Resistance Associated Protein 4 (MRAP-4).

As the time of exposure increased, RPTEC/TERT1 cells exposed to flow shear stress began to express more and different genes compared to cells cultures under static conditions. Table II below lists the RPTEC/TERT1 genes whose expression was significantly increased or decreased in rotating spinpod cultures compared to static spinpod cultures at the 3 hour, 24 hour, and 72 hour time points.

TABLE II
Genes that differed significantly between rotating and
static spinpod cultures of hTERT at 3, 24, or 72 hours

| | | | | | | | | |
|---|---|---|---|---|---|---|---|---|
| | | | | log2 | | | | |
| | | Spin | Stat | (fold | Test | P | Q | |
| gene | locus | FPKM | FPKM | change) | stat | value | value | Role |

INCREASED WITH ROTATION AT 3 HRS

| | | | | | | | | |
|---|---|---|---|---|---|---|---|---|
| ATP6V0A1 | chr17:42458843- | 258.7 | 12.9 | −4.3 | −26.4 | 5E−05 | 0.0076 | ATPase H+ Transporting V0 |
| | 42522579 | | | | | | | Subunit A1 |

DECREASED WITH ROTATION AT 24 HRS.

| | | | | | | | | |
|---|---|---|---|---|---|---|---|---|
| SERPINB2 | chr18:63887704- | 0.1 | 0.9 | 2.8 | 2.5 | 7E−04 | 0.0403 | Serpin Family B Member 2 |
| | 63903890 | | | | | | | |
| NPTX1 | chr17:80466832- | 0.2 | 1.3 | 2.6 | 4.2 | 5E−05 | 0.0052 | neuronal pentraxin gene family |
| | 80476604 | | | | | | | |
| RRM2 | chr2:10122567- | 3.7 | 16.5 | 2.1 | 5.7 | 5E−05 | 0.0052 | Ribonucleotide Reductase |
| | 10131419 | | | | | | | Regulatory Subunit M2 |

INCREASED WITH ROTATION AT 24 HRS.

| | | | | | | | | |
|---|---|---|---|---|---|---|---|---|
| AKR1B10 | chr7:134527591- | 15.8 | 3.3 | −2.3 | −5.3 | 5E−05 | 0.0052 | Aldo-Keto Reductase Family |
| | 134541414 | | | | | | | |
| ARNT2 | chr15:80404349- | 48.3 | 3.8 | −3.7 | −13.1 | 5E−05 | 0.0052 | Aryl Hydrocarbon Receptor |
| | 80597936 | | | | | | | Nuclear Translocator 2 |

DECREASED WITH ROTATION AT 72 HOURS

| | | | | | | | | |
|---|---|---|---|---|---|---|---|---|
| RPL7 | chr8:73290638- | 2.6 | 32.8 | 3.6 | 6.3 | 5E−05 | 0.0048 | ribosomal protein |
| | 73293634 | | | | | | | |
| MKI67 | chr10:128096660- | 0.1 | 0.9 | 3.0 | 4.9 | 5E−05 | 0.0048 | Marker Of Proliferation Ki-67 |
| | 128126204 | | | | | | | |
| CRISPLD2 | chr16:84819980- | 0.5 | 3.7 | 3.0 | 5.3 | 5E−05 | 0.0048 | Cysteine Rich Secretory Protein |
| | 84909510 | | | | | | | |
| DLGAP5 | chr14:55148115- | 0.2 | 1.4 | 2.9 | 3.6 | 5E−05 | 0.0048 | DLG Associated Protein 5 |
| | 55191678 | | | | | | | |
| TTC29 | chr4:146707026- | 0.2 | 1.2 | 2.7 | 2.4 | 8E−04 | 0.0321 | Tetratricopeptide Repeat |
| | 146945882 | | | | | | | Domain 9 |
| PTN | chr7:137227345- | 0.4 | 2.5 | 2.7 | 3.2 | 5E−05 | 0.0048 | Pleiotrophin |
| | 137343800 | | | | | | | |
| AMTN | chr4:70518571- | 1.4 | 8.9 | 2.6 | 3.9 | 5E−05 | 0.0048 | Amelotin calcium phosphate |
| | 70532743 | | | | | | | mineralization |
| CYP1A1 | chr15:74719541- | 0.2 | 1.0 | 2.6 | 2.7 | 4E−04 | 0.0209 | aryl hydrocarbon hydroxylase |
| | 74725536 | | | | | | | |
| RRM2 | chr2:10122567- | 0.7 | 4.0 | 2.5 | 4.3 | 5E−05 | 0.0048 | Ribonucleotide Reductase |
| | 10131419 | | | | | | | Regulatory Subunit M2 |
| TOP2A | chr17:40388520- | 1.0 | 5.1 | 2.4 | 5.2 | 5E−05 | 0.0048 | DNA Topoisomerase II Alpha |
| | 40417950 | | | | | | | |
| FGF1 | chr5:142592177- | 0.1 | 0.7 | 2.3 | 2.5 | 5E−05 | 0.0048 | Fibroblast growth factor-1 |
| | 142698070 | | | | | | | |
| SHCBP1 | chr16:46580555- | 0.4 | 1.9 | 2.3 | 3.4 | 5E−05 | 0.0048 | SHC Binding And Spindle |
| | 46621399 | | | | | | | Associated 1 |
| KIAA0101 | chr15:64365011- | 1.0 | 4.8 | 2.2 | 3.2 | 5E−05 | 0.0048 | PCNA-associated factor |
| | 64387687 | | | | | | | |
| EDN2 | chr1:41478774- | 1.1 | 5.0 | 2.2 | 2.9 | 1E−04 | 0.0084 | Endothelin 2 |
| | 41484683 | | | | | | | |
| PTCHD2 | chr1:11479237- | 0.2 | 0.8 | 2.2 | 2.7 | 3E−04 | 0.0167 | Dispatched 3 gene |
| | 11537583 | | | | | | | |
| NOTCH2NL | chr1:146151907- | 1.0 | 4.5 | 2.2 | 4.6 | 5E−05 | 0.0048 | Notch homolog 2 N-terminal-like |
| | 146229032 | | | | | | | |
| RTKN2 | chr10:62193085- | 0.3 | 1.5 | 2.2 | 2.5 | 1E−04 | 0.0084 | Rhotekin 2 |
| | 62268863 | | | | | | | |
| ASPM | chr1:197084126- | 0.2 | 0.9 | 2.2 | 3.0 | 5E−05 | 0.0048 | Abnormal spindle-like |
| | 197146694 | | | | | | | microcephaly-associated |
| ASF1B | chr19:14119508- | 0.7 | 2.9 | 2.1 | 2.9 | 5E−05 | 0.0048 | Anti-Silencing Function 1B |
| | 14136628 | | | | | | | Histone Chaperone |
| PCDH19 | chrX:100291643- | 0.2 | 0.7 | 2.1 | 3.0 | 5E−05 | 0.0048 | protocadherin 19 |
| | 100410273 | | | | | | | |
| EXO1 | chr1:241848190- | 0.2 | 0.9 | 2.1 | 2.6 | 2E−04 | 0.0115 | 5′ to 3′ exonuclease |
| | 241889939 | | | | | | | |
| CSF2 | chr5:132073791- | 1.1 | 4.8 | 2.1 | 2.5 | 7E−04 | 0.0309 | Colony Stimulating Factor 2 |
| | 132076170 | | | | | | | |
| KIF4A | chrX:70290028- | 0.1 | 0.6 | 2.1 | 2.4 | 8E−04 | 0.0321 | Kinesin Family Member 4A |
| | 70420924 | | | | | | | |
| GJB2 | chr13:20187464- | 0.5 | 1.9 | 2.0 | 2.8 | 5E−05 | 0.0048 | gap junction beta 2 |
| | 20192975 | | | | | | | |
| FAM111B | chr11:59107184- | 0.2 | 0.7 | 2.0 | 2.3 | 7E−04 | 0.0318 | Family With Sequence Similarity |
| | 59127416 | | | | | | | 111 Member B |
| ADAMTS15 | chr11:130448973- | 0.2 | 1.0 | 2.0 | 2.9 | 5E−05 | 0.0048 | ADAM Metallopeptidase |
| | 130476644 | | | | | | | |
| MYBL2 | chr20:43667018- | 0.6 | 2.5 | 2.0 | 3.3 | 5E−05 | 0.0048 | Myb-related protein; cell |
| | 43716496 | | | | | | | progression |
| NUF2 | chr1:163321932- | 0.3 | 1.2 | 2.0 | 2.3 | 1E−03 | 0.0400 | Kinetochore protein Nuf2 |
| | 163355763 | | | | | | | |

INCREASED WITH ROTATION AT 72 HRS.

| | | | | | | | | |
|---|---|---|---|---|---|---|---|---|
| DUSP15 | chr20:31861066- | 28.0 | 6.9 | −2.0 | −3.8 | 5E−05 | 0.0048 | Dual Specificity Phosphatase 15 |
| | 31870676 | | | | | | | |
| RN7SL2 | chr14:49862550- | 171.4 | 37.5 | −2.2 | −2.9 | 5E−05 | 0.0048 | signal recognition particle |
| | 49862849 | | | | | | | |
| HSPA1B | chr6_GL000251 | 1.9 | 0.4 | −2.2 | −3.0 | 5E−05 | 0.0048 | Heat Shock Protein Family B; |
| | v2_alt:3304987- | | | | | | | hsp70 |
| | 3307510 | | | | | | | |
| HSPA1B | chr6_GL000250 | 21.4 | 4.4 | −2.3 | −5.1 | 5E−05 | 0.0048 | Heat Shock Protein Family B; |
| | v2_alt:3160359- | | | | | | | hsp71 |
| | 3162873 | | | | | | | |
| CRYAB | chr11:111908619- | 58.5 | 12.1 | −2.3 | −4.6 | 5E−05 | 0.0048 | Crystallin Alpha B |
| | 111926871 | | | | | | | |
| SLC30A2 | chr1:26038021- | 1.2 | 0.2 | −2.7 | −2.8 | 3E−04 | 0.0167 | zinc transporter |
| | 26046138 | | | | | | | |
| HSPA6 | chr1:161524539- | 13.7 | 2.1 | −2.7 | −5.6 | 5E−05 | 0.0048 | Heat Shock Protein Family A |
| | 161526897 | | | | | | | (Hsp70) Member 6 |
| | | | | | | | | mediates insertion secretory |
| RN7SL1 | chr14:49586579- | 182.3 | 27.1 | −2.7 | −4.3 | 5E−05 | 0.0048 | proteins into ER |
| | 49586878 | | | | | | | |
| RN7SK | chr6:52995619- | 207.0 | 28.0 | −2.9 | −4.6 | 5E−05 | 0.0048 | RNA Gene, |
| | 52995951 | | | | | | | |
| SNORD3A | chr17:19188015- | 167.7 | 13.0 | −3.7 | −2.8 | 5E−05 | 0.0048 | Small Nucleolar RNA |
| | 19188232 | | | | | | | |
| RPPH1 | chr14:20343070- | 33.7 | 2.6 | −3.7 | −3.6 | 5E−05 | 0.0048 | RNase P ribonucleoprotein |
| | 20343411 | | | | | | | |

The quantity of thirteen cytokines/chemokines were measured in the supernatants of RPTEC/TERT1 after 72 hours in rotating and static spinpods: IL1β, IL-2, IL-4, IL-5, IL-6, IL-8, IL-10, IL-12, IL-13, GM-CSF, IFNγ, MCP-1, and TNFα. Four of these were present in significantly different quantities in the supernatant of PCT exposed to rotation compared to static cultures (Table III and FIG. 6). GM-CSF was 0.43±0.02 fold lower (p<0.0001), IL-6 was 0.68±0.09 fold lower (p=0.03), MCP-1 was 0.32±0.09 fold-lower, and IL-12 was 0.71±0.12 fold-lower (p=0.04).

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A suspension culture device comprising:
a rotatable base having an exterior surface that engages at least one roller for rotation of the base about an axis when the at least one roller is turning;
first and second end components attached to the base along the axis, wherein the base and the first and second end components define an interior space for holding liquid, wherein a portion of at least one of the end components is made of a material that is at least partially transparent for viewing into the interior space from outside the base; and
a plurality of ports that each permit fluid communication between the interior space and outside the base.

2. The suspension culture device of claim 1, wherein at least a portion of the base is made of one of fluoroplastic, fluorinated ethylene propylene (FEP), PerFluoroAlkoxy (PFA), and polytetrafluoroetylene (PTFE).

3. The suspension culture device of claim 1, wherein the base is substantially shaped as a disk.

4. The suspension culture device of claim 1, wherein the base includes an outer edge that rotates about the axis.

5. The suspension culture device of claim 4, wherein the ports are positioned at the outer edge.

6. The suspension culture device of claim 4, wherein the ports include a first port and a second port, and wherein the first port and the second port are positioned at substantially opposing portions of the outer edge.

7. The suspension culture device of claim 4, wherein the ports include a first port, a second port, a third port, and a fourth port, wherein the first port and the second port are in proximity to each other, wherein the third port and the fourth port are in proximity to each other, and wherein the first and second ports are positioned at substantially opposing portions of the outer edge from the third and fourth ports.

8. The suspension culture device of claim 1, wherein the number of ports is three or more.

9. The suspension culture device of claim 1, wherein the ports are each made of a silicone rubber material.

10. The suspension culture device of claim 1, wherein the interior space is substantially shaped as a disk.

11. The suspension culture device of claim 1, wherein the interior space has a volume between about 0.3 milliliters and about 250 milliliters.

12. The suspension culture device of claim 1, further comprising a plurality of windows attached to the base for permitting viewing into the interior space.

13. The suspension culture device of claim 1, wherein the rotatable base defines an outer edge that is substantially round for contact with one or more rollers for rotating the rotatable base.

14. The suspension culture device of claim 1, wherein the culture includes a cell culture medium and cells.

15. The suspension culture device of claim 14, wherein the interior space is configured to hold one of support structures, beads, test substances, drugs, peptides, and viruses.

16. The suspension culture device of claim 1, further comprising (tab) elements to facilitate one of robotic assembly, robotic loading, refeeding, unloading, and injecting drugs or biologics.

17. A suspension culture system comprising:
at least one roller;
a mechanism configured to turn the at least one roller; and
a suspension culture device comprising:
a rotatable base having an exterior surface that engages the at least one roller for rotation of the base about an axis when the at least one roller is turning;
first and second end components attached to the base along the axis, wherein the base and the first and second end components define an interior space for holding liquid, wherein a portion of at least one of the end components is made of a material that is at least partially transparent for viewing into the interior space from outside the base; and
a plurality of ports that each permit fluid communication between the interior space and outside the base.

18. The system of claim 17, wherein at least a portion of the base is made one of fluoroplastic, fluorinated ethylene propylene (FEP), PerFluoroAlkoxy (PFA), and polytetrafluoroetylene (PTFE).

19. The system of claim 17, wherein the rotatable base is substantially shaped as a disk.

20. The system of claim 17, wherein the rotatable base includes an axis of rotation and an outer edge that rotates about the axis.

21. The system of claim 20, wherein the ports are positioned at the outer edge.

22. The system of claim 20, wherein the rotatable base is at least partially transparent.

23. The system of claim 20, wherein the ports include a first port and a second port, and wherein the first port and the second port are positioned at substantially opposing portions of the outer edge.

24. The suspension culture device of claim 20, wherein the ports include a first port, a second port, a third port, and a fourth port, wherein the first port and the second port are in proximity to each other, wherein the third port and the fourth port are in proximity to each other, and wherein the first and second ports are positioned at substantially opposing portions of the outer edge from the third and fourth ports.

25. The system of claim 17, wherein the ports are each made of a silicone rubber material.

26. The system of claim 17, wherein the interior space is substantially shaped as a disk.

27. The system of claim 17, wherein the interior space has a volume between about 0.3 milliliters and about 250 milliliters.

28. The system of claim 17, wherein the suspension culture device further comprises a plurality of windows attached to the base for permitting viewing into the interior space.

29. The system of claim 17, wherein the rotatable base defines an outer edge that is substantially round for contact with one or more rollers for rotating the rotatable base.

30. The system of claim 17, wherein the culture includes a cell culture medium and cells.

31. The system of claim 30, wherein the interior space is configured to hold one of support structures, beads, test substances, drugs, peptides, bacteria, algae, fungi, and viruses.

32. The system of claim 17, wherein the suspension culture device further comprises (tab) elements to facilitate one of robotic assembly, robotic loading, refeeding, unloading, and injecting drugs or biologics.

33. An adaptor for holding a suspension culture device for observation of contents of the suspension culture device, the adaptor comprising:
a base portion comprising a top portion defining a surface and a bottom portion defining a surface, the base portion defining an aperture that extends between the surface of the top portion and the surface of the bottom portion; and
a suspension culture device holder comprising a first feature and a second feature, the first feature configured for holding a suspension culture device, and the second feature configure for fitting to the aperture of the base portion.

34. The adaptor of claim 33, wherein the holder defines an aperture that aligns with the aperture of the base portion when the suspension culture device holder is fitted to the aperture of the base portion.
